(19) United States
(12) Patent Application Publication
(10) Pub. No.: US 2013/0209366 A1
(43) Pub. Date: Aug. 15, 2013

(54) Glutathione-Lanthionine Compounds and Methods Related Thereto

(71) Applicant: Kenneth Hensley, Sylvania, OH (US)

(72) Inventor: Kenneth Hensley, Sylvania, OH (US)

(73) Assignees: OKLAHOMA MEDICAL RESEARCH FOUNDATION, Oklahoma City, OK (US); UNIVERSITY OF TOLEDO, Toledo, OH (US)

(21) Appl. No.: 13/818,439

(22) PCT Filed: Aug. 24, 2011
(86) PCT No.: PCT/US11/48940

§ 371 Date: Mar. 27, 2013

Related U.S. Application Data
(60) Provisional application No. 61/376,566, filed on Aug. 24, 2010.

(51) Int. Cl. C07K 5/02; A61K 38/07; A61K 31/19; A61K 45/06

(52) U.S. Cl. CPC C07K 5/0215; A61K 45/06; A61K 38/07; A61K 31/19
USPC 424/9.2; 530/323; 514/1.3; 435/29

(57) ABSTRACT

The present invention concerns glutathione-lanthionine compounds, the process of preparing such compounds, and their use. The invention also concerns methods of using the compounds and derivatives and combinations of these compounds in the treatment and/or prevention diseases, including diseases affecting the central nervous system.

### CROSS-REFERENCE TO RELATED APPLICATIONS

This is a national stage application filed under 37 CFR 1.371 of international application PCT/US20xx/xxxxxx filed xxx, xx, xxxx which claims the priority to U.S. Provisional Application Ser. No. 61/376,566 filed Aug. 24, 2010, the entire disclosures of which are expressly incorporated herein by reference.

### STATEMENT OF FEDERAL SPONSORSHIP

This invention was made with no Governmental support.

### FIELD OF THE INVENTION

The present invention concerns glutathione-lanthionine compounds, the process of preparing such compounds, and their use. The invention also concerns methods of using the compounds and derivatives and combinations of these compounds in the treatment and/or prevention diseases, including diseases affecting the central nervous system.

### BACKGROUND OF THE INVENTION

The nonproteogenic aminio acid lanthionine (Lan, or alanine-5-alanine) along with some of its derived metabolites, particularly the cyclic thioether lanthionine ketimine (LK; 2H-1,4-thiazine-5,6-dihydro-3,5-dicarboxylic acid) are present in mammalian central nervous system (CNS).

A pyroxidal phosphate-dependent enzyme, GTK/KAT1, catalyzes the transamination of Lan with α-keto acids such as pyruvate (FIG. 2). The products of this chemistry include an unstable intermediate that rapidly cyclizes to yield a thioether ketimine. When Lan is the aminoacyl substrate for the transamination, the product is specifically lanthionine ketimine (LK) (FIGS. 2-3). Other substrates besides Lan are acceptable (e.g., cystathionine, thialysine), giving rise to a family of unusual cyclic thioethers that are demonstrably present in mammalian brain [12]. The same products can be formed through a mechanism catalyzed by amino acid oxidase (AAO), however the transaminase activity is probably a more significant pathway in the mammalian brain owing to the relatively high specific activity of brain GTK/KAT1 over AAO [12-14].

GTK/KAT1 activity is has been shown to catalyze conversion of the tryptophan oxidation product, kynurenine, into the cyclic product kynurenic acid (KYNA) (FIG. 3) [15]. KYNA is now established to be an endogenous anti-excitotoxin that binds to an allosteric site on NMDA receptors to antagonize excessive glutamate neurotransmission [15-17]. There is some evidence that endogenous KYNA regulates sensitivity of striatal neurons to quinolate, which has implications to the etiology of Huntington's disease (HD), a condition in which these neurons are particularly vulnerable [18]. Furthermore, mutations in KAT 1 are common features of spontaneously hypertensive rat (SHR) strains [19] such that direct administration of kynurenic acid to the rostral ventrolatral medulla (RVLM) can temporarily alleviate hypertension in these animals [20]. Thus, KYNA is indicated as an endogenous paracrine substance formed by action of kynurenine pathway enzymes upon tryptophan metabolites, with important physiological functions still being elucidated. It is therefore conceivable that LK and its molecular family members, formed from analogous action of GTK/KAT1 (FIG. 4), serve analogous paracrine functions to KYNA.

Using HPLC and gas-liquid chromatography, Cavallini and colleagues measured LK in mammalian brain at concentrations near 1 nmole/g tissue [12, 21-22]. Recognizing that the brain is frugal with respect to conservation of reduced sulfur, Cavallini thought it highly unlikely that evolution would have failed to close such a wasteful sulfur “leak” unless lanthionine served a useful biochemical function. Despite significant research effort Cavallini never discovered specific bioactivities for LK, however he did report that [35S]LK bound syntaptosomal membranes tightly (Kd=58 nM, in the range of typical neurotransmitter affinities) [23]. [35S]LK binding was saturable and reversible with cold LK, suggesting a receptor:ligand interaction [12,23]. The binding was specific because reduced LK (thiomorpholine dicarboxylic acid) could not displace [35S]LK [12,23].

U.S. Pat. No. 7,683,055 discloses a library of useful LK molecules and related methods. U.S. Pat. No. 7,683,055 is explicitly incorporated by reference, in its entirety, to this application.

Fontana [23] stated that LK might bind selectively to brain proteins, but candidate proteins were not identified.

In 2007, the present inventor and collaborators lab identified lanthionine synthetase-like protein-1 (LanCL1) as a prominently-expressed brain protein that strongly bound both GSH and GSSG (but not other sulfurous amino acids such as cysteine) and which was over-expressed in spinal cords of SOD1G93A mice [31]. LanCL1 subsequently was crystallized with bound GSH and the X-ray structure published [32].

In 2010, the present inventor and collaborators used mass spectrometry-assisted protein microsequencing techniques to screen a mammalian brain proteome for LK binding partners [24]. LK was coupled to a solid phase support by Mannich chemistry, and exposed to solubilized bovine brain extract. Free LK was used to selectively elute LK-bound species. Three proteins emerged that appeared to bind LK selectively but were not likely to bind nonselectively to mock-treated solid phase supports. These were: collapsing response mediator protein-2 (CRMP2; also known as DRP2 or DYPSL2); syntaxin-binding protein-1 (STXBP1, also known as Munc18 or nSec1); and lanthionine synthetase-like protein 1 (LanCL1) [24].

These three proteins are becoming recognized for their roles in neurite outgrowth, synaptogenesis, and neurotransmission. CRMP2 is crucial to mediate growth factor-dependent axon and dendrite growth and regulates neuron polarization (the process by which one neurite becomes an axon) during embryonic neurogenesis [29]. STXBP1/Munc-18 is part of the presynaptic protein machinery that regulates fusion of pre-formed neurotransmitter vesicles with the plasma membrane during neurotransmitter docking and exocytosis [30]. LanCL1 is a particularly intriguing protein that also binds GSH [31-32]. Cellular functions of LanCL1 and its homolog LanCL2 are still being elucidated, but mutants of LanCL1 appear to act in a dominant negative fashion to interfere with growth factor-dependent neurite outgrowth in neuroculture [32]. The relevance of both CRMP2 and LanCL1 to neuritigenesis therefore provide a basis for the observed neurotrophic or neuritigenic effects of LK.

As yet, LanCL1/2 have no reported enzymatic functions, but are sequence homologs of bacterial lanthionine cyclase (LanC) enzymes which catalyze regioselective, intramolecular conjugation of protein Cys residues to nearby, dehydrated Thr or Ser residues within specific precursor polypeptides. In most cases the relevant Ser or Thr is dehydrated to dehydroalanine or dehydrobutyrine, respectively, by a separate dehydratase which does not have a mammalian homolog. The resulting product of multiple LanC catalyses is a polymacrocyclic polypeptide termed a lantibiotic [33-34]. Lantibiotics are amongst the most potent antibiotic substances yet discovered, and as such, have generated tremendous research interest amongst microbiologist and medicinal chemists [33-34]. Both LanC and LanCL1 have a structurally similar Zn²⁺ binding domain which in LanC is the catalytic pocket for the enzyme. Glutathione binds in the LanCL1 version of this pocket, tightly associating with specific amino acid residues to constrain GSH orientation while allowing the central thiol to ligate the Zn²⁺ metallocenter [32]. Ample space exists within the GSH binding pocket of LanCL1 to permit co-occupancy of another molecule, but as of yet no co-substrate has been identified for GSH conjugation by LanCL1.

Recent studies are now uncovering demonstrable bioactivities inherent to LK that are manifest at low microM concentrations in cell culture systems [24] or in vivo. The inventor synthesized cell permeable LK pro-drugs including hydrolysable LK-5-ethyl ester (LKE) from reaction of 3-bromopyruvate with L-cysteine ethyl ester [24-25]. Both LK and LKE suppressed nitric oxide synthesis in tumor necrosis factor-α (TNFα) or lipopolysaccharide (LPS)-treated microglia and macrophages, with the ester being more potent [24-25]. LKE protected NSC-34 motor neuron-like cells from toxicity of hydrogen peroxide (H₂O₂) [24-25] and from toxicity of microglia-conditioned medium [25]. LK and LKE also protected HT-4 neurons against toxicity of high-dose glutamate, though in this case LK was more potent [25]. Most notably, LKE promoted the growth factor-stimulated outgrowth of neurites in NSC-34 cells [24-25] which could have broad implications to the use of synthetic LK derivatives or prodrugs for the treatment of neurodegenerative conditions [24-25].

### SUMMARY OF THE INVENTION

The present invention provides improved compounds, compositions and methods related to a precursor/prodrug of LK. For example, the present invention provides glutathione (GSH) derivatives composed of GSH sulfur heteroatoms covalently bonded to the β-carbon atom of an alanine, to form a central lanthionine. Such compositions are designated herein as glutathione lanthionine derivatives, or “gLan compounds.”

An exemplary glutathione-lanthionine chimera (i.e. glutathione-S-alanine or “gLan”) is shown in FIG. 7). The inventor has synthesized gLan through the reaction of GSH with β-chloroalanine and actively investigated its chemical and biological properties and potential as a novel medicament. The gLan compounds were are predicted to be a metabolic precursor for LK and display bioactivities similar to those of LK.

The gLan compositions diminish inflammatory cell activation, promote neurite outgrowth, slow neurodegeneration in a variety of diseases. Such diseases include amyotrophic lateral sclerosis (ALS) and Alzheimer's disease, among others. Additional methods related to use of gLan against behavioral disorders such as schizophrenia are also within the scope of the present invention. The gLan compositions herein are useful for the same purposes as the LK derivatives discussed in U.S. Pat. No. 7,683,055 but have the advantages of being more chemically stable, are more capable of crossing the blood-brain barrier, and demonstrate greater efficacy in a mouse model of ALS than LK-ethyl ester (LKE).

The present invention therefore provides gLan compounds, compositions and derivatives, the process of preparing such compounds, and their use. More particularly, it concerns methods of using gLan compounds for the treatment and/or prevention diseases, including diseases affecting the central nervous system, such as amyotrophic lateral sclerosis. The invention provides for compounds and methods having anti-oxidant, anti-neuroinflammatory and neuroprotective activities. Furthermore, the invention provides compounds that show anti-proliferative effects and may therefore be useful for the treatments of cancer.

The present invention provides compounds having the structure of Formula I:

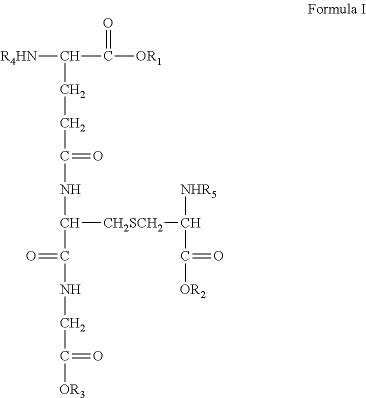

wherein R₁, R₂ and R₃ are each independently selected from the group consisting of hydrogen, halogen, alkyl, alkenyl, alkynyl, cycloalkyl, cycloalkenyl, substituted alkyl, substituted alkenyl, substituted alkynyl, substituted cycloalkyl, substituted cycloalkenyl, aryl, heteroaryl silyl, heterosilyl, and heterocyclic; wherein R₄ and R₅ are each independently selected from the group consisting of —H; —C(═O)—CH₃; —C(═O)—X—NH₂, wherein X is a heteroatom substituted or unsubstituted alkyl or alkynyl, or heteroatom-substituted CN-amido.; and pharmaceutically acceptable salts, hydrates, and optical isomers thereof.

Therefore, provided are those compounds wherein R₁, R₂ and R₃ are each —OH.

Therefore, provided are those compounds wherein R₄ and R₅ are each —NH₂.

Therefore, provided are those compounds wherein R₁, R₂ and R₃ are each —OH and wherein R₄ and R₅ are each —NH₂.

Also provided are compositions of matter comprising γGlu-Cys(S-Ala)-Gly, and pharmaceutically acceptable salts or hydrates thereof.

Also provided are methods of treating a disease comprising: administering to a subject a pharmacologically effective amount of a compound described herein.

Therefore, provided are methods wherein the subject is a mammal.

Therefore, provided are methods wherein the subject is a human.

Therefore, provided are methods wherein the method further comprises administering a second anti-inflammatory compound, adjuvant or additional therapeutic to the subject.

Therefore, provided are methods wherein the second anti-inflammatory compound is pyruvate.

Therefore, provided are methods wherein the disease is an inflammatory disease.

Therefore, provided are methods wherein the disease is selected from the group consisting of: sepsis; amyotrophic lateral sclerosis (ALS), a degenerative motor neuron disease, Alzheimer's disease, Parkinson's disease, Huntington's disease, multiple sclerosis, macular degeneration, a cardiovascular disease, atherosclerosis, rheumatoid arthritis; lupus; fibromyalgia; neuropathy; allergy; autoimmune disease; diabetes; ulcerative colitis; or inflammatory bowel disease (IBD) hypertension, attention deficit disorder, depression (e.g., major depression); schizophrenia; chronic pain; or generalized anxiety disorder.

Also provided are methods for evaluating the effectiveness of a compound of claim 1 as useful for treatment of inflammatory diseases, wherein the method comprises: (i) introducing a compound described herein to a macrophage cell, and (ii) measuring the response of the macrophage cell to an inflammatory stimulus.

Therefore, provided are methods wherein the macrophage cell is a microglial cell.

Therefore, provided are methods wherein the inflammatory stimulus is a pro-inflammatory cytokine.

Therefore, provided are methods wherein the pro-inflammatory cytokine is TNFα or IFNγ.

Therefore, provided are methods wherein nitric oxide production from the macrophage cell is evaluated.

Therefore, provided are methods wherein said evaluation comprises measuring nitrite production from the macrophage cell.

Also provided are methods for evaluating the effectiveness of a compound described herein as useful for treatment of inflammatory diseases, wherein the method comprises: (i) introducing a compound as described herein to a mouse model of inflammation, and (ii) measuring the response of the mouse model to an inflammatory stimulus.

Therefore, provided are methods wherein the mouse model is SOD1^{G93A} mouse.

Therefore, provided are methods wherein the inflammatory stimulus is a pro-inflammatory cytokine.

Therefore, provided are methods wherein the pro-inflammatory cytokine is TNFα or IFNγ.

Therefore, provided are methods wherein onset of paralysis is measured.

Therefore, provided are methods wherein age at death is measured.

Also provided are methods of reducing damage to a cell resulting from oxidative stress and/or excitatory amino acid toxicity, wherein the compound is contacted with the cell, wherein the cell is selected from the group consisting of: a neuron, a macrophage or a glial cell; motoneuron; astroglia cell; a microglial cell, wherein the glial cell is not a glioma cell.

Therefore, provided are methods wherein the cell is present in a subject.

Therefore, provided are methods, wherein the subject is a human patient.

Also provided are methods of treating a subject at risk for having a stroke, comprising administering to the subject a pharmacologically effective amount of the compound described herein.

Therefore, provided are methods wherein the subject is a human patient.

Therefore, provided are methods wherein the subject has had a stroke.

Also provided are methods of treating a subject with cancer, comprising administering to the subject a pharmacologically effective amount of a compound as described herein.

Therefore, provided are methods wherein said cancer is brain, lung, liver, spleen, kidney, lymph node, small intestine, pancreas, blood cell, bone, colon, stomach, bread, endometrium, prostate, testicle, ovary, central nervous system, skin, head and neck, esophagus, or bone marrow cancer.

Various aspects of this invention will become apparent to those skilled in the art from the following detailed description of the preferred embodiment, when read in light of the accompanying drawings.

### BRIEF DESCRIPTION OF THE FIGURES

The patent or application file contains one or more drawings executed in color and/or one or more photographs. Copies of this patent or patent application publication with color drawing(s) and/or photograph(s) will be provided by the Patent Office upon request and payment of the necessary fee.

FIG. 1. The classic transulfuration pathway.

FIG. 2. CβS catalysis of lanthionine formation and its processing through a kynurenine pathway “shunt”.

FIG. 3. A: Structure and numbering convention for lanthionine ketimine (LK) tautomers and synthetic derivatives. For natural lanthionine ketimine, R₁═R₂═H. B: Tautomerism between the imine and ene-amine forms of LK.

FIG. 4. The classic kynurenine pathway of tryptophan metabolism, onto which is superimposed a proposed “shunt” through which lanthionine is metabolized to LK via the action of GTK/KAT1 (blue labels). Pyr=pyruvate; other α-keto acids can substitute. αKB=α-ketobutyrate.

FIG. 5. LK-5-ethyl ester (LKE) promotes neurite extension in primary, dissociated chick dorsal root ganglia cell cultures. Neurons were treated with LKE or saline vehicle for 24 h from DIV2-3 and quantitatively assessed for neurite morphometry as described in the text.

FIG. 6. LKE slows progression of paralytic disease in the SOD1^{G93A} mouse model of familial amyotrophic lateral sclerosis (ALS). Rotarod performance curves are shown for mice injected intraperitoneally with 100 mg/kg/d LKE, beginning at 90d. Bars represent mean±SEM; *p<0.05 by post-hoc two-tailed t-test.

FIG. 7. Summary of possible routes for enzymatic formation of lanthionine via either the transulfuration and kynurenine pathways or from glutathione-lanthionine (gLan).

FIG. 8. Treatment with synthetic gLan improves motor function in SOD1^{G93A} mice. Mice were treated with daily intraperitoneal injections of gLan, at the indicated dose, 5 days/week beginning at 90 d, and motor performance was evaluated by rotarod task as described in the examples.

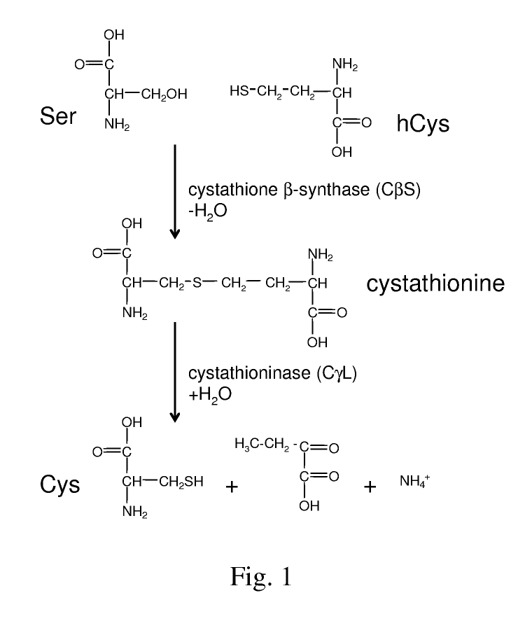

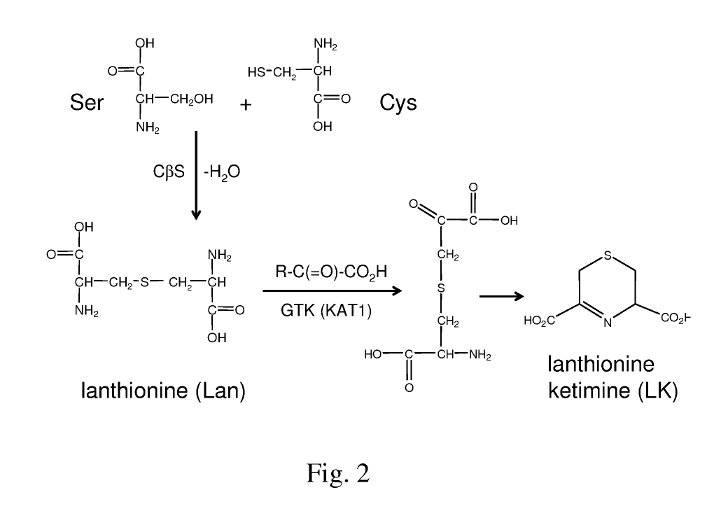

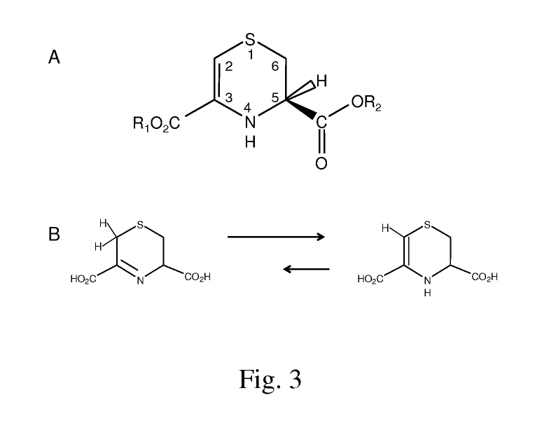

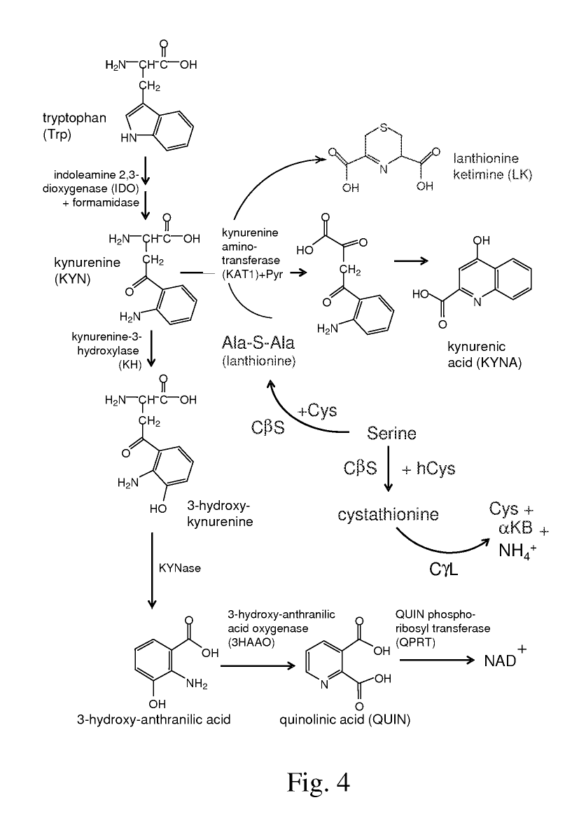

### DETAILED DESCRIPTION OF THE INVENTION

The significance of lanthionines in paracrine signaling provides to opportunities for utilizing bioavailable pro-drugs of these compounds as novel pharmacophores. Lanthionine (Lan), the mono-sulfide analog of cystine, is a natural but nonproteogenic amino acid thought to form in mammals mainly through promiscuous reactivity of the transulfuration enzyme cystathionine-β-synthase (CβS). Lanthionine exists at appreciable concentrations in mammalian brain, where it undergoes aminotransferase conversions mediated by kynurenine pathway enzymes to yield unusual cyclic thioether ketimines. Recently one of these Lan metabolites, lanthionine ketimine (LK; 2H-1,4-thiazine-5,6-dihydro-3,5-dicarboxylic acid) was discovered to possess neuroprotective, neuritigenic and anti-inflammatory activities.

In some of these embodiments, the functional group interacts with blood-brain barrier (BBB)-specific transport mechanisms. For example, ascorbyl derivatives of the compounds are be expected to take advantage of BBB ascorbyl transporters. Also, certain amino acid esters or amide derivatives readily transport the compound across the BBB by means of BBB transport enzymes. Methods of making ascorbyl, dehydroascorbyl, and amino acid esters of drugs containing carboxylic acids are well-known in the art. Conjugation of ascorbyl, dehydroascorbyl, serinyl, or glycinyl to the compound may be performed using techniques known in the art. See e.g., Manfredini et al., 2001 and Huang et al., 2001.

The practice of the present invention will employ, unless otherwise indicated, conventional techniques of cell biology, cell culture, molecular biology, microbiology, recombinant DNA, medicine, pharmacology and immunology, which are within the skill of the art. Such techniques are explained fully in the literature. See, for example, Sambrook et al., 1989; Ausubel et al., 1994; Glover, 1985; Gait, 1984; U.S. Pat. No. 4,683,195; Hames and Higgins, 1985; Mayer and Walker, 1988; Weir and Blackwell, 1986.

### DEFINITIONS

The terms “inhibiting,” “reducing,” or “prevention,” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an,” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions of the invention can be used to achieve methods of the invention.

Throughout this application, the term “about” is used to indicate that a value includes the inherent variation of error for the device, the method being employed to determine the value, or the variation that exists among the study subjects.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As Used Herein,

“Alkyl” refers to monovalent alkyl groups preferably having from 1 to 10 carbon atoms and more preferably 1 to 6 carbon atoms. This term is exemplified by groups such as methyl, ethyl, n-propyl, iso-propyl, n-butyl, iso-butyl, n-hexyl, and the like.

“Substituted alkyl” refers to an alkyl group, preferably of from 1 to 10 carbon atoms, having from 1 to 3 substituents selected from the group consisting of alkoxy, substituted alkoxy, acyl, acylamino, amino, aminoacyl, aminocarboxy esters, cyano, cycloalkyl, halogen, hydroxyl, carboxyl, carboxylalkyl, oxyacyl, oxyacylamino, thiol, thioalkoxy, substituted thioalkoxy, aryl, heteroaryl, heterocyclic, aryloxy, thioaryloxy, heteroaryloxy, thioheteroaryloxy, nitro, and mono- and di-alkylamino, mono- and di-(substituted alkyl)amino, mono- and di-arylamino, mono- and di-heteroarylamino, mono- and di-heterocyclic amino, and unsymmetric di-substituted amines having different substituents selected from alkyl, substituted alkyl, aryl, heteroaryl and heterocyclic.

“Alkylene” refers to divalent alkylene groups preferably having from 1 to 10 carbon atoms and more preferably 1 to 6 carbon atoms. This term is exemplified by groups such as methylene (—CH₂—), ethylene (—CH₂CH₂—), the propylene isomers (e.g., —CH₂CH₂CH₂— and —CH(CH₃)CH₂—), and the like.

“Alkaryl” refers to -alkylene-aryl groups preferably having from 1 to 10 carbon atoms in the alkylene moiety and from 6 to 10 carbon atoms in the aryl moiety. Such alkaryl groups are exemplified by benzyl, phenethyl and the like.

“Alkoxy” refers to the group “alkyl-O—”. Preferred alkoxy groups include, by way of example, methoxy, ethoxy, n-propoxy, iso-propoxy, n-butoxy, tert-butoxy, sec-butoxy, n-pentoxy, n-hexoxy, 1,2-dimethylbutoxy, and the like.

“Substituted alkoxy” refers to the group “substituted alkyl-O—” where substituted alkyl is as defined above.

“Alkenyl” refers to alkenyl groups preferably having from 2 to 10 carbon atoms and more preferably 2 to 6 carbon atoms and having at least 1 and preferably from 1-2 sites of alkenyl unsaturation. Preferred alkenyl groups include ethenyl (—CH═CH₂), n-propenyl (—CH₂CH═CH₂), iso-propenyl (—C(CH₃)═CH₂), but-2-enyl (—CH₂CH═CHCH₃), and the like.

“Substituted alkenyl” refers to an alkenyl group as defined above having from 1 to 3 substituents selected from the group consisting of alkoxy, substituted alkoxy, acyl, acylamino, amino, aminoacyl, aminocarboxy esters, cyano, halogen, hydroxyl, carboxyl, carboxylalkyl, cycloalkyl, oxyacyl, oxyacylamino, thiol, thioalkoxy, substituted thioalkoxy, aryl, heteroaryl, heterocyclic, nitro, and mono- and di-alkylamino, mono- and di-(substituted alkyl)amino, mono- and di-arylamino, mono- and di-heteroarylamino, mono- and di-heterocyclic amino, and unsymmetric di-substituted amines having different substituents selected from alkyl, substituted alkyl, aryl, heteroaryl and heterocyclic.

“Alkenylene” refers to divalent alkenylene groups preferably having from 2 to 8 carbon atoms and more preferably 2 to 6 carbon atoms. This term is exemplified by groups such as ethenylene (—CH≡H—), the propenylene isomers (e.g., —CH₂CH═CH— and —C(CH₃)═CH—) and the like.

“Substituted alkenylene” refers to an alkenylene group, preferably of from 2 to 8 carbon atoms, having from 1 to 3 substituents selected from the group consisting of alkoxy, substituted alkoxy, acyl, acylamino, acyloxy, amino, aminoacyl, aminoacyloxy, cyano, halogen, hydroxyl, carboxyl, carboxylalkyl, thiol, thioalkoxy, substituted thioalkoxy, aryl, heteroaryl, heterocyclic, nitro, and mono- and di-alkylamino, mono- and di-(substituted alkyl)amino, mono- and di-arylamino, mono- and di-heteroarylamino, mono- and di-heterocyclic amino, and unsymmetric di-substituted amines having different substituents selected from alkyl, substituted alkyl, aryl, heteroaryl and heterocyclic. Additionally, such substituted alkylene groups include those where 2 substituents on the alkylene group are fused to form one or more cycloalkyl, aryl, heterocyclic or heteroaryl groups fused to the alkylene group.

“Alkynyl” refers to alkynyl groups preferably having from 2 to 10 carbon atoms and more preferably 2 to 6 carbon atoms and having at least 1 and preferably from 1-2 sites of alkynyl unsaturation. Preferred alkynyl groups include ethynyl (—≡CH), propargyl (—CH₂≡CH) and the like.

“Substituted alkynyl” refers to an alkynyl group as defined above having from 1 to 3 substituents selected from the group consisting of alkoxy, substituted alkoxy, acyl, acylamino, amino, aminoacyl, aminocarboxy esters, cyano, halogen, hydroxyl, carboxyl, carboxylalkyl, cycloalkyl, oxyacyl, oxyacylamino, thiol, thioalkoxy, substituted thioalkyoxy, aryl, heteroaryl, heterocyclic, nitro, and mono- and di-alkylamino, mono- and di-(substituted alkyl)amino, mono- and di-arylamino, mono- and di-heteroarylamino, mono- and di-heterocyclic amino, and unsymmetric di-substituted amines having different substituents selected from alkyl, substituted alkyl, aryl, heteroaryl and heterocyclic.

“Aryl” refers to an unsaturated aromatic carbocyclic group of from 6 to 14 carbon atoms having a single ring (e.g., phenyl) or multiple condensed rings (e.g., naphthyl or anthryl). Preferred aryls include phenyl, naphthyl and the like.

Unless otherwise constrained by the definition for the aryl substituent, such aryl groups can optionally be substituted with from 1 to 3 substituents selected from the group consisting of hydroxy, acyl, alkyl, alkoxy, alkenyl, alkynyl, substituted alkyl, substituted alkoxy, substituted alkenyl, substituted alkynyl, amino, aminoacyl, aminocarboxy esters, alkaryl, aryl, aryloxy, carboxyl, carboxylalkyl, acylamino, cyano, halo, nitro, heteroaryl, heterocyclic, oxyacyl, oxyacylamino, thioalkoxy, substituted thioalkoxy, trihalomethyl, mono- and di-alkylamino, mono- and di-(substituted alkyl)amino, mono- and di-arylamino, mono- and di-heteroarylamino, mono- and di-heterocyclic amino, and unsymmetric di-substituted amines having different substituents selected from alkyl, substituted alkyl, aryl, heteroaryl and heterocyclic, and the like. Preferred substituents include alkyl, alkoxy, halo, cyano, nitro, trihalomethyl, and thioalkoxy.

“Aryloxy” refers to the group aryl-O— wherein the aryl group is as defined above including optionally substituted aryl groups as also defined above.

“Carboxyalkyl” refers to the groups —C(O)O-alkyl and —C(O)O-substituted alkyl where alkyl and substituted alkyl are as defined above.

“Cycloalkyl” refers to cyclic alkyl groups of from 3 to 8 carbon atoms having a single cyclic ring or multiple condensed rings which can be optionally substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, or multiple ring structures such as adamantanyl, and the like.

“Substituted cycloalkyl” refers to cycloalkyl groups having from 1 to 5 (preferably 1 to 3) substituents selected from the group consisting of hydroxy, acyl, acyloxy, alkyl, substituted alkyl, alkoxy, substituted alkoxy, alkenyl, substituted alkenyl, alkynyl, substituted alkynyl, amino, aminoacyl, alkaryl, aryl, aryloxy, carboxyl, carboxylalkyl, cyano, halo, nitro, heteroaryl, thioalkoxy, substituted thioalkoxy, trihalomethyl and the like.

“Cycloalkenyl” refers to cyclic alkenyl groups of from 4 to 8 carbon atoms having a single cyclic ring and at least one point of internal unsaturation which can be optionally substituted with from 1 to 3 alkyl groups. Examples of suitable cycloalkenyl groups include, for instance, cyclobut-2-enyl, cyclopent-3-enyl, cyclooct-3-enyl and the like.

“Substituted cycloalkenyl” refers to cycloalkenyl groups having from 1 to 5 substituents selected from the group consisting of hydroxy, acyl, acyloxy, alkyl, substituted alkyl, alkoxy, substituted alkoxy, alkenyl, substituted alkenyl, alkynyl, substituted alkynyl, amino, aminoacyl, alkaryl, aryl, aryloxy, carboxyl, carboxylalkyl, cyano, halo, nitro, heteroaryl, thioalkoxy, substituted thioalkoxy, trihalomethyl and the like.

“Halo” or “halogen” refers to fluoro, chloro, bromo and iodo and preferably is either chloro or bromo.

“Heteroaryl” refers to a monovalent aromatic carbocyclic group of from 2 to 10 carbon atoms and 1 to 4 heteroatoms selected from oxygen, nitrogen and sulfur within the ring.

Unless otherwise constrained by the definition for the heteroaryl substituent, such heteroaryl groups can be optionally substituted with 1 to 3 substituents selected from the group consisting of hydroxy, acyl, alkyl, alkoxy, alkenyl, alkynyl, substituted alkyl, substituted alkoxy, substituted alkenyl, substituted alkynyl, amino, aminoacyl, aminocarboxy esters, alkaryl, aryl, aryloxy, carboxyl, carboxylalkyl, aminoacyl, cyano, halo, nitro, heteroaryl, heterocyclic, oxyacyl, oxyacylamino, thioalkoxy, substituted thioalkoxy, trihalomethyl, mono- and di-alkylamino, mono- and di-(substituted alkyl)amino, mono- and di-arylamino, mono- and di-heteroarylamino, mono- and di-heterocyclic amino, and unsymmetric di-substituted amines having different substituents selected from alkyl, substituted alkyl, aryl, heteroaryl and heterocyclic, and the like. Preferred substituents include alkyl, alkoxy, halo, cyano, nitro, trihalomethyl, and thioalkoxy. Such heteroaryl groups can have a single ring (e.g., pyridyl or furyl) or multiple condensed rings (e.g., indolizinyl or benzothienyl). Preferred heteroaryls include pyridyl, pyrrolyl and furyl.

“Heterocycle” or “heterocyclic” refers to a monovalent saturated or unsaturated group having a single ring or multiple condensed rings, from 1 to 8 carbon atoms and from 1 to 4 hetero atoms selected from nitrogen, sulfur or oxygen within the ring.

Unless otherwise constrained by the definition for the heterocyclic substituent, such heterocyclic groups can be optionally substituted with 1 to 3 substituents selected from the group consisting of hydroxy, acyl, alkyl, alkoxy, alkenyl, alkynyl, substituted alkyl, substituted alkoxy, substituted alkenyl, substituted alkynyl, amino, aminoacyl, aminocarboxy esters, alkaryl, aryl, aryloxy, carboxyl, carboxylalkyl, aminoacyl, cyano, halo, nitro, heteroaryl, heterocyclic, oxyacyl, oxyacylamino, thioalkoxy, substituted thioalkoxy, trihalomethyl, mono- and di-alkylamino, mono- and di-(substituted alkyl)amino, mono- and di-arylamino, mono- and di-heteroarylamino, mono- and di-heterocyclic amino, and unsynumetric di-substituted amines having different substituents selected from alkyl, substituted alkyl, aryl, heteroaryl and heterocyclic, and the like. Such heterocyclic groups can have a single ring or multiple condensed rings. Preferred heterocyclics include morpholino, piperidinyl, and the like.

Examples of heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholino, piperidinyl, pyrrolidine, tetrahydrofuranyl, and the like.

As used herein, the term “amino” means —NH2; the term “nitro” means —NO2; the term “halo” designates —F, —Cl, —Br or —I; the term “mercapto” means —SH; the term “cyano” means —CN; the term “silyl” means —SiH3, and the term “hydroxy” means —OH.

The term “heteroatom-substituted,” when used to modify a class of organic radicals (e.g. alkyl, aryl, acyl, etc.), means that one, or more than one, hydrogen atom of that radical has been replaced by a heteroatom, or a heteroatom containing group. Examples of heteroatoms and heteroatom containing groups include: hydroxy, cyano, alkoxy, ═O, ═S, —NO₂, —N(CH₃)₂, amino, or —SH. Specific heteroatom-substituted organic radicals are defined more fully below.

The term “heteroatom-unsubstituted,” when used to modify a class of organic radicals (e.g. alkyl, aryl, acyl, etc.) means that none of the hydrogen atoms of that radical have been replaced with a heteroatom or a heteroatom containing group. Substitution of a hydrogen atom with a carbon atom, or a group consisting of only carbon and hydrogen atoms, is not sufficient to make a group heteroatom-substituted. For example, the group —C₆H₄≡CH is an example of a heteroatom-unsubstituted aryl group, while —C₆H₄F is an example of a heteroatom-substituted aryl group. Specific heteroatom-unsubstituted organic radicals are defined more fully below.

The term “heteroatom-unsubstituted Cn-alkyl” refers to a radical, having a linear or branched, cyclic or acyclic structure, further having no carbon-carbon double or triple bonds, further having a total of n carbon atoms, all of which are nonaromatic, 3 or more hydrogen atoms, and no heteroatoms. For example, a heteroatom-unsubstituted C₁-C₁₀-alkyl has 1 to 10 carbon atoms. The term “alkyl” includes straight-chain alkyl groups, branched-chain alkyl groups, cycloalkyl (alicyclic) groups, alkyl heteroatom-substituted cycloalkyl groups, and cycloalkyl heteroatom-substituted alkyl groups. The groups, —CH₃, —CH₂CH₃, —CH₂CH₂CH₃, —CH(CH₃)₂, —CH(CH₂)₂, —CH₂CH₂CH₂CH₃, —CH(CH₃)CH₂CH₃, —CH₂CH(CH₃)₂, —C(CH₃)₃, —CH₂C(CH₃)₃, cyclopentyl, and cyclohexyl, are all examples of heteroatom-unsubstituted alkyl groups.

The term “heteroatom-substituted Cn-alkyl” refers to a radical, having a single saturated carbon atom as the point of attachment, no carbon-carbon double or triple bonds, further having a linear or branched, cyclic or acyclic structure, further having a total of n carbon atoms, all of which are nonaromatic, 0, 1, or more than one hydrogen atom, at least one heteroatom, wherein each heteroatom is independently selected from the group consisting of N, O, F, Cl, Br, I, Si, P, and S. For example, a heteroatom-substituted C₁-C₁₀-alkyl has 1 to 10 carbon atoms. The following groups are all examples of heteroatom-substituted alkyl groups: trifluoromethyl, —CH₂F, —CH₂Cl, —CH₂Br, —CH₂OH, —CH₂OCH₃, —CH₂OCH₂CH₃, —CH₂OCH₂CH₂CH₃, —CH₂OCH(CH₃)₂, —CH₂OCH(CH₂)₂, —CH₂OCH₂CF₃, —CH₂OCOCH₃, —CH₂NH₂, —CH₂NHCH₃, —CH₂N(CH₃)₂, —CH₂NHCH₂CH₃, —CH₂N(CH₃)CH₂CH₃, —CH₂NHCH₂CH₂CH₃, —CH₂NHCH(CH₃)₂, —CH₂NHCH(CH₂)₂, —CH₂N(CH₂CH₃)₂, —CH₂CH₂F, —CH₂CH₂Cl, —CH₂CH₂Br, —CH₂CH₂I, —CH₂CH₂OH, CH₂CH₂OCOCH₃, —CH₂CH₂NH₂, —CH₂CH₂N(CH₃)₂, —CH₂CH₂NHCH₂CH₃, —CH₂CH₂N(CH₃)CH₂CH₃, —CH₂CH₂NHCH₂CH₂CH₃, —CH₂CH₂NHCH(CH₃)₂, —CH₂CH₂NHCH(CH₂)₂, —CH₂CH₂N(CH₂CH₃)₂, —CH₂CH₂NHCO₂C(CH₃)₃, and —CH₂Si(CH₃)₃.

The term “heteroatom-unsubstituted Cn-alkenyl” refers to a radical, having a linear or branched, cyclic or acyclic structure, further having at least one nonaromatic carbon-carbon double bond, but no carbon-carbon triple bonds, a total of n carbon atoms, three or more hydrogen atoms, and no heteroatoms. For example, a heteroatom-unsubstituted C₂-C₁₀-alkenyl has 2 to 10 carbon atoms. Heteroatom-unsubstituted alkenyl groups include: —CH═CH₂, —CH═CHCH₃, —CH═CHCH₂CH₃, —CH═CHCH₂CH₂CH₃, —CH═CHCH(CH₃)₂, —CH═CHCH(CH₂)₂, —CH₂CH═CH₂, —CH₂CH═CHCH₃, —CH₂CH═CHCH₂CH₃, —CH₂CH═CHCH₂CH₂CH₃, —CH₂CH═CHCH(CH₃)₂, —CH₂CH═CHCH(CH₂)₂, and —CH═CH—C₆H₅.

The term “heteroatom-substituted Cn-amido” refers to a radical, having a single nitrogen atom as the point of attachment, further having a carbonyl group attached via its carbon atom to the nitrogen atom, further having a linear or branched, cyclic or acyclic structure, further having a total of n aromatic or nonaromatic carbon atoms, 0, 1, or more than one hydrogen atom, at least one additional heteroatom in addition to the oxygen of the carbonyl group, wherein each additional heteroatom is independently selected from the group consisting of N, O, F, Cl, Br, I, Si, P, and S. For example, a heteroatom-substituted C₁-C₁₀-amido has 1 to 10 carbon atoms. The term “heteroatom-substituted Cn-amido” includes groups, having the structure —NHR, in which R is a heteroatom-unsubstituted Cn-acyl, as that term is defined above. The group, —NHCO₂CH₃, is an example of a heteroatom-substituted amido group.

The term “pharmaceutically acceptable salts,” as used herein, refers to salts of compounds of this invention that are substantially non-toxic to living organisms. Typical pharmaceutically acceptable salts include those salts prepared by reaction of a compound of this invention with an inorganic or organic acid, or an organic base, depending on the substituents present on the compounds of the invention.

Examples of inorganic acids which may be used to prepare pharmaceutically acceptable salts include: hydrochloric acid, phosphoric acid, sulfuric acid, hydrobromic acid, hydroiodic acid, phosphorous acid and the like. Examples of organic acids which may be used to prepare pharmaceutically acceptable salts include: aliphatic mono- and dicarboxylic acids, such as oxalic acid, carbonic acid, citric acid, succinic acid, phenyl-heteroatom-substituted alkanoic acids, aliphatic and aromatic sulfuric acids and the like. Pharmaceutically acceptable salts prepared from inorganic or organic acids thus include hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydrofluoride, acetate, propionate, formate, oxalate, citrate, lactate, p-toluenesulfonate, methanesulfonate, maleate, and the like. Other suitable salts are known to one of ordinary skill in the art.

Suitable pharmaceutically acceptable salts may also be formed by reacting the agents of the invention with an organic base such as methylamine, ethylamine, ethanolamine, lysine, ornithine and the like. Other suitable salts are known to one of ordinary skill in the art.

Pharmaceutically acceptable salts include the salts formed between carboxylate or sulfonate groups found on some of the compounds of this invention and inorganic cations, such as sodium, potassium, ammonium, or calcium, or such organic cations as isopropylammonium, trimethylammonium, tetramethylammonium, and imidazolium.

It should be recognized that the particular anion or cation forming a part of any salt of this invention is not critical, so long as the salt, as a whole, is pharmacologically acceptable and as long as the anion or cation does not contribute undesired qualities or effects. Further, additional pharmaceutically acceptable salts are known to those skilled in the art, and may be used within the scope of the invention. Additional examples of pharmaceutically acceptable salts and their methods of preparation and use are presented in Pharmaceutical Salts: Properties, Selection and Use—A Handbook, by C. G. Wermuth and P. H. Stahl, Verlag Helvetica Chimica Acta, 2002, which is incorporated herein by reference.

As used herein, the term “patient” is intended to include living organisms in which certain conditions as described herein can occur. Examples include humans, monkeys, cows, sheep, goats, dogs, cats, mice, rats, and transgenic species thereof. In a preferred embodiment, the patient is a primate. In an even more preferred embodiment, the primate is a human. Other examples of subjects include experimental animals such as mice, rats, dogs, cats, goats, sheep, pigs, and cows. The experimental animal can be an animal model for a disorder, e.g., a transgenic mouse with an Alzheimer's-type neuropathology. A patient can be a human suffering from a neurodegenerative disease, such as Alzheimer's disease, or Parkinson's disease.

As used herein, the term “IC50” refers to an inhibitory dose which is 50% of the maximum response obtained.

As used herein, the term “water soluble” means that the compound dissolves in water at least to the extent of 0.010 mole/liter or is classified as soluble according to literature precedence.

As used herein, “predominantly one enantiomer” means that the compound contains at least 95% of one enantiomer, or more preferably at least 98% of one enantiomer, or most preferably at least 99% of one enantiomer. Similarly, the phrase “substantially free from other optical isomers” means that the composition contains at most 5% of another enantiomer or diastereomer, more preferably 2% of another enantiomer or diastereomer, and most preferably 1% of another enantiomer or diastereomer.

Other abbreviations used herein are as follows: DMSO, dimethyl sulfoxide; iNOS, inducible nitric oxide synthase; COX-2, cyclooxygenase-2; NGF, nerve growth factor; IBMX, isobutylmethylxanthine; FBS, fetal bovine serum; GPDH, glycerol 3-phosphate dehydrogenase; RXR, retinoid X receptor; TGF-β, transforming growth factor-β; IFN-γ, interferon-γ; LPS, bacterial endotoxic lipopolysaccharide; TNF-α, tumor necrosis factor-α; IL-1β, interleukin-1β; GAPDH, glyceraldehyde-3-phosphate dehydrogenase; MTT, 3-[4,5-dimethylthiazol-2-yl]-2,5-diphenyltetrazolium bromide; TCA, trichloroacetic acid; HO-1, inducible heme oxygenase.

Pharmaceutical Preparations

Pharmaceutical compositions of the present invention comprise an effective amount of a compound(s) or composition(s) disclosed herein, and/or additional agents, dissolved or dispersed in a pharmaceutically acceptable carrier. The phrases “pharmaceutical” or “pharmacologically acceptable” refers to molecular entities and compositions that produce no adverse, allergic or other untoward reaction when administered to an animal, such as, for example, a human. The preparation of a pharmaceutical composition that contains at least one compound or additional active ingredient will be known to those of skill in the art in light of the present disclosure, as exemplified by Remington's Pharmaceutical Sciences, 2003, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 995, pp. 1289-1329, incorporated herein by reference). Except insofar as any conventional carrier is incompatible with the active ingredient, its use in the pharmaceutical compositions is contemplated.

A composition of the present invention, may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 2003, incorporated herein by reference).

A composition of the present invention may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, composition(s) of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the pharmaceutical composition may include small quantities of pharmacologically acceptable chelators or co-antioxidants. Examples of chelators include ethylenediaminetetraacetic acid (EDTA) and ethylene glycol-bis(beta-aminoethyl ether)-N,N,N′,N′-tetraacetic acid (EGTA). Examples of antioxidants including gallate esters, ascorbate, vitamin E (or other tocopherols), butylated hydroxytoluene, and/or benzoic acid. These chelators and/or co-antioxidants may be used to stabilize a composition of the present invention. In certain embodiments, these chelators and/or antioxidants may stabilize a composition of the present invention, from decomposition by autooxidation.

In further embodiments, the present invention may concern the use of a pharmaceutical lipid vehicle that includes a composition of the present invention, one or more lipids, and an aqueous solvent. As used herein, the term “lipid” will be defined to include any of a broad range of substances that is characteristically insoluble in water and extractable with an organic solvent. This broad class of compounds is known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, a composition of the present invention may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

Alimentary Compositions and Formulations

In certain embodiments of the present invention, a composition herein, and/or additional agents is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Parenteral Compositions and Formulations

In further embodiments, a composition of the present invention, may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,753,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 nil of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

Miscellaneous Pharmaceutical Compositions and Formulations

In other preferred embodiments of the invention, the active compound may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch.” For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

Methods of Use

The methods and compounds of the present invention may be used for prevention and treatment of cancer, diseases involving inflammation and/or oxidative stress, and/or disorders of the central nervous system (CNS), including stroke. In certain embodiments, the invention provides methods of treating and/or preventing a disease or disorder of the central nervous system, such as those mentioned above, or throughout this application, in an individual comprising, administering at least one compound of this invention to the individual in an amount effective to treat and/or prevent the disease.

The present invention provides methods for treating a disease (e.g., an inflammatory disease) in an individual comprising administering a composition of the present invention to the individual in amount of a composition of the present invention effective to treat the disease. In certain embodiments a specific optical isomer of a composition of the present invention is used. Optionally, a composition of the present invention may be administered in combination with other compounds, such a lanthionine, a compound of U.S. Pat. No. 7,683,055, glucose, pyruvate, or an ester or amide derivative of pyruvate to stimulate endogenous production of LK.

The principle and mode of operation of this invention have been explained and illustrated in its preferred embodiment. However, it must be understood that this invention may be practiced otherwise than as specifically explained and illustrated without departing from its spirit or scope

### EXAMPLES

### Example 1

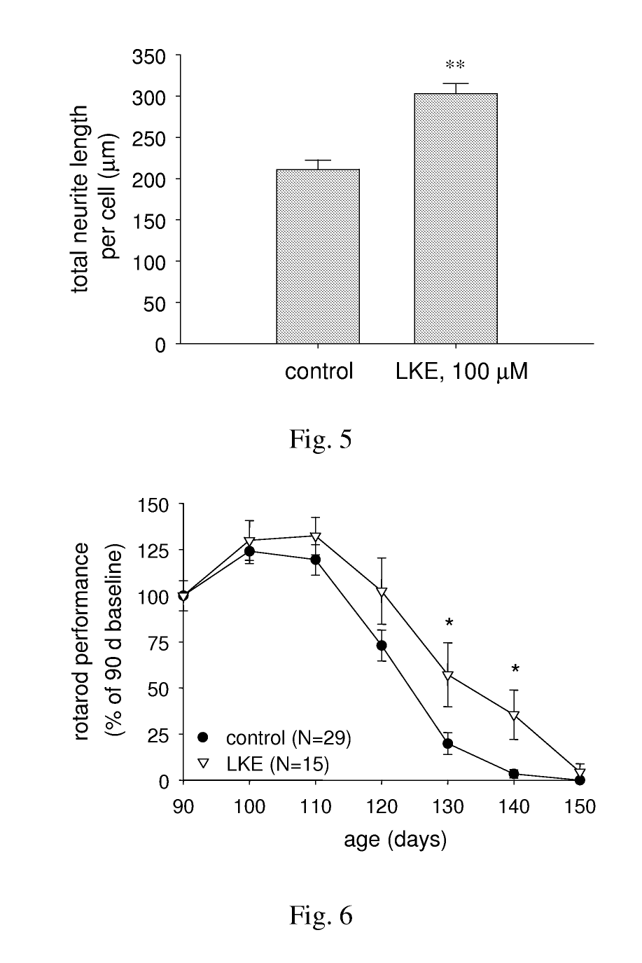

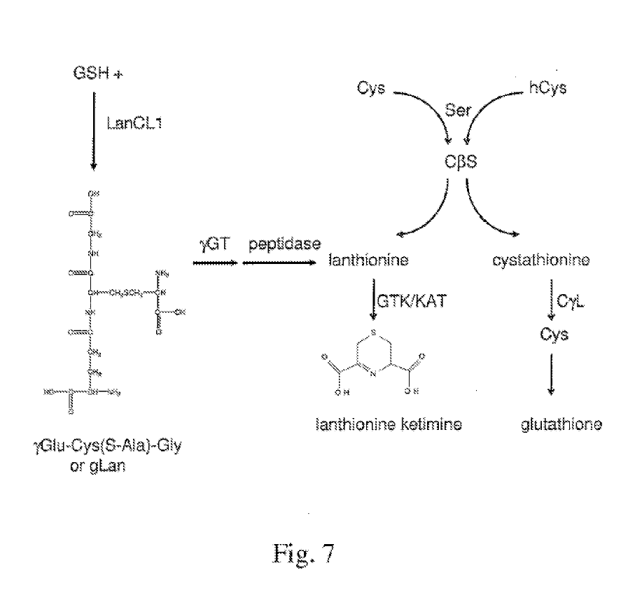

Cell culture studies were performed to assess LKE effects on neuritigenesis in primary dissociated chick dorsal root ganglia (DRG) cultures prepared essentially as described by Margiotta and Howard [26]. Cultures were treated with LKE at 2-3 days in vitro (DIV), and neuron morphometry was quantified from bright field micrographs by a blinded observer using Metamorph® software (Molecular Devices, Sunnyvale Calif. USA). At 100 μM, LKE significantly increased the mean neurite length of dissociated DRG neurons (FIG. 5), similar to effects observed previously in NSC-34 cultures [24].

### Example 2

Additional studies to explore effects of LKE in vivo, as a pharmacological candidate were conducted in a murine model for the motoneuron disease amyotrophic lateral sclerosis (ALS). In a first effort to assess LKE effects against a model of spontaneous neurodegeneration, SOD1G93A mutant mice modeling familial ALS [27-28] were treated with intraperitoneal (i.p.) injections of 100 mg/kg/d LKE in saline vehicle, from 90d throughout the remaining mouse lifespan. LKE is relevant to test in this mouse because the SOD1G93A mouse experiences robust neuroinflammation concomitant with TNFα-driven microglial activation, oxidative stress, and significant progressive distal axonopathy [27], all of which might be expected to be mitigated by LKE based on the ester's observed effects in cell culture. Mouse motor function was assessed as previously described using a standard rotarod test [27-28], at sequential 10d intervals (FIG. 6). Specific groups of mice were treated with LKE in saline or saline vehicle only. As illustrated in FIG. 6 and Table I, LKE significantly slowed progression of clinical motoneuron disease [25], mostly through delaying onset of clinical paralysis as defined by a clinical leg-splay test [27-28]. Overall survival was increased by LKE based on logrank analysis (Table I). These findings suggest that further research is justified to determine the potential of bioavailable LK derivatives or pro-drugs for the treatment of neurodegenerative pathologies.

TABLE I
Effects of LKE on clinical parameters in SOD1^{G93A} mice.
Drug was administered intraperitoneally, in saline,
at 100 mg/kg/d beginning at 90d. data are mean ± SEM.

| | vehicle (N = 29) | LKE (N = 15) |
|---|---|---|
| age of frank paralysis (d) | 113.7 ± 1.4 | 121.6 ± 2.5* |
| median age at death (d) | 132 | 139† |
| minimum age at death (d) | 117 | 124 |
| maximum age at death (d) | 142 | 152 |

*p,0.05 by t-test;
†0.05 by log-rank test.

### Example 3

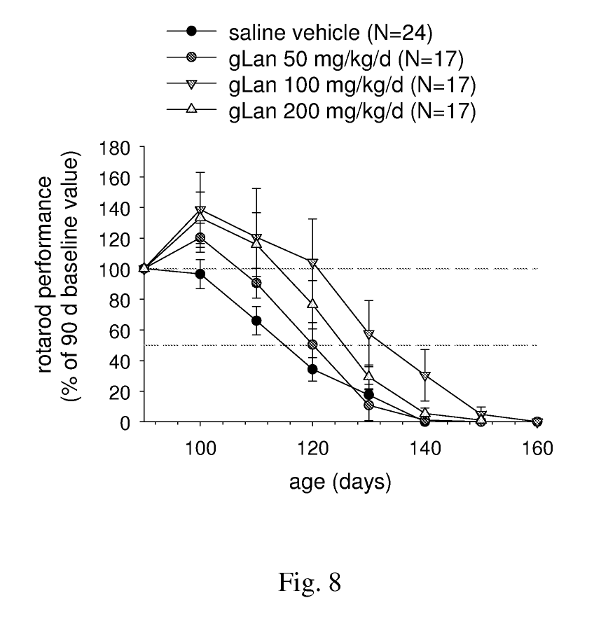

Mice were injected with synthetic gLan in saline at 50-200 mg/kg/d, five days per week (Monday-Friday) beginning at 90d of age. Mice were assessed at baseline and every 10d for motor functional ability using the rotarod task as described previously [27-28]. As illustrated in FIG. 8 and Table II, gLan dose-dependently improved motor function in SOD1G93A mice up to 110d or 120d with 100 mg/kg being optimum (p<0.01 for overall gLan effect based on repeated measures ANOVA).

TABLE II
Effects of gLan upon onset-of-paralysis and age-at-death parameters
in SOD1^{G93A} mice treated with the indicated daily dose of gLan
beginning at 90d as indicated in text and FIG. 8.

| | | onset of paralysis (d) | | age at death (d) | |
|---|---|---|---|---|---|
| gLan dose | | mean ± SD | | mean ± SD | |
| vehicle | | 112.8 | ± 5.4 | 128.6 | ± 9.6 |
| 50 | mg/kg | 111.6 | ± 3.8 | 126.7 | ± 6.9 |
| 100 | mg/kg | 123.8 | ± 4.9* | 138.3 | ± 9.5* |
| 200 | mg/kg | 116.7 | ± 5.1 | 133.1 | ± 9.3 |

*p,0.02 by two-tailed t-test.

Interestingly, average rotarod performance at 100d and 110d in mice treated with 100-200 mg/kg/d gLan, actually improved above the 90d baseline value (FIG. 8). Transgenic animals treated with these doses of gLan displayed, on average, twice the motor function as saline treated control animals at ages <120d (FIG. 8). Motor function dropped off rapidly amongst all groups at ages >110 d (FIG. 8). As in the case of LKE treatment, chronic intraperitoneal gLan at 100-200 mg/kg/dose tended to increase the age at which onset-of-paralysis was observed (based on a leg-clinch test [28]) and the mean age of death of the SOD1G93A mice (Table II). When motor performance curves were interpolated to estimate the age at which mice lost an average 50% of baseline motor function, the 100 mg/kg/dose gLan treatment group reached this clinical endpoint approximately 18d later than the vehicle-treated group (FIG. 8). The 100 mg/kg gLan dose appeared generally more effective than the 200 mg/kg dose, suggesting there may be a therapeutic dosage window.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

The publication and other material used herein to illuminate the invention or provide additional details respecting the practice of the invention, are incorporated be reference herein, and for convenience are provided in the following bibliography.

Citation of the any of the documents recited herein is not intended as an admission that any of the foregoing is pertinent prior art. All statements as to the date or representation as to the contents of these documents is based on the information available to the applicant and does not constitute any admission as to the correctness of the dates or contents of these documents.

### REFERENCES

1. Mechanism of Sulfur Reactions. William A. Pryor, Ed., McGraw-Hill Book Co. Inc, New York N.Y. USA, 1962.
2. Biological interactions of sulfur compounds. Stephen C. Mitchell, Ed., Taylor and Francis Publishers, Bristol Pa. USA, 1996.
3. Meister, A.; Anderson, M. E. Glutathione. Annu. Rev. Biochem. 1983, 52, 711-760.
4. Awasthi, Y. C.; Chaudhary, P.; Vatsyayan, R.; Sharma, A.; Awasthi, S.; Sharma, R. Physiological and pharmacological significance of glutathione-conjugate transport. J. Toxicol. Environ. Health B Crit. Rev. 2009, 12, 540-541.
5. Mieyall, J. J.; Gallogly, M. M.; Qanungo, S.; Sabens, E. A.; Shelton, M. D. Molecular mechanisms and clinical implications of reversible protein S-glutathionylation. Antioxid. Redox Signal. 2008, 10, 1941-1988.
6. Gabbita, S. P.; Robinson, K. A.; Stewart, C. A.; Floyd, R. A.; Hensley, K. Redox regulatory mechanisms of cellular signal transduction. Arch. Biochem. Biophys. 2000, 376, 1-13.
7. Stryer, L.; Biochemistry 3^{rd} Ed. W.H. Freeman and Company; New York, USA; 1988.
8. Singh, R. K.; Gupta, S.; Dastidar, S.; Ray, A. Cysteinyl leukotrienes and their receptors: Molecular and functional characteristics. Pharmacol. 2010, 85, 336-349.
9. Obeid, R.; Herrmann, W. Mechanisms of homocysteine neurotoxicity in neurodegenerative diseases with special reference to dementia. FEBS Lett. 2006, 580, 2994-3005.
10. Braunstein, A. E.; Goryachenkova, E. V. The beta-replacement-specific pyridoxal-P-dependent lyases. Adv. Enzymol. Relat. Areas Mol. Biol., 1984, 56, 1-89.
11. Cavallini, D.; Ricci, G.; Federri, G. The ketimine derivatives of thialysine, lanthionine, cystathionine, cysteine: Preparation and properties. 1983, In Sulfur Amino Acids: Biochemical and Clinical Aspects, Alan R. Liss Inc., pp. 355-364.
12. Cavallini, D.; Ricci, G.; Dupre, S.; Pecci, L.; Costa, M.; Matarese, R. M.; Pensa, B.; Antonuci, A.; Solinas, S. P.; Fontana, M. Sulfur-containing cyclic ketimines and imino acids. A novel family of endogenous products in search for a role. Eur J. Biochem., 1991, 202, 217-223.
13. Cooper, A. J.; Anders, M. W. Glutamine transaminase K and cysteine conjugate □-lyase. Ann. NY Acad. Sci. 1990, 585, 118-127.
14. Cooper, A. J. L. The role of glutamine transaminase K (GTK) in sulfur and alpha-keto acid metabolism in the brain, and in the possible bioactivation of neurotoxicants. Neurochem Int. 2004, 44, 557-577.
15. Moroni, F. Tryptophan metabolism and brain function: Focus on kynurenine and other indole metabolites. Eur. J. Pharmacol. 1999, 1375, 87-100.
16. Foster, A. C.; Kemp, J. A.; Leeson, P. D.; Grimwood, S.; Donald, A. E.; Marshall, G. R.; Priestley, T.; Smith, J. D.; Carling, P. W. Kynurenic acid analogs with improved affinity and selectivity for the glycine site at the N-methyl-D-aspartate receptor from rat brain. Mol. Pharmacol. 1992, 41, 910-922.
17. Urenjak, J.; Obrenovitch, T. P. Neuroprotective potency of kynurenic acid against excitotoxicity. NeuroReport 2000, 11, 1341-1344.
18. Sapko, M. T.; Guidetti, P.; Yu, P.; Tagle, D. A.; Pellicciari, R.; Schwarcz, R. Endogenous kynurenate controls the vulnerability of striatal neurons to quinolinate: Implications for Huntington's disease. Exp. Neurol. 2006, 197: 4-7.
19. Kwok, J. B.; Kapoor, R.; Gotoda, T.; Iwamoto, Y.; Lizuka, Y.; Yamada, N.; Isaacs, K. E.; Kushwaha, V. V.; Church, W. B.; Schofield, P. R.; Kapoor, V. A missense mutation in kynurenine aminotransferase-1 in spontaneously hypertensive rats. J. Biol. Chem. 2002, 277, 35779-35782.
20. Ito, S.; Komatsu, K.; Tsukamoto, K.; Sved, A. F. Excitatory amino acids in the rostral ventrolateral medulla support blood pressure in spontaneously hypertensive rats. Hypertension, 2000, 35, 413-417.
21. Ricci, G.; Vesci, L.; Nardini, M.; Arduini, A.; Storto, S.; Rosato, N.; Cavallini, D. Detection of 2H-1,4-thiazine-5,6-dihydro-3,5-dicarboxylic acid (lanthionine ketimine) in the bovine brain by a fluorometric assay. Biochim. Biophys. Acta, 1989, 990, 211-215.
22. Fontana, M.; Brunori, A.; Costa, M.; Antonucci, A. Detection of cystathionine ketimine and lanthionine ketimine in human brain. Neurochem. Res. 1997, 22, 821-844.
23. Fontana, M.; Ricci, G.; Solinas, S. P.; Antonucci, A.; Serao, I.; Dupre, S.; Cavallini, D. ^{[35}S^{]} Lanthionine ketimine binding to bovine brain membranes. Biochem. Biophys. Res. Commun. 1990, 171, 480-486.
24. Hensley, K.; Christov, A.; Kamat, S.; Zhang, X. C.; Jackson, K. W.; Snow, S.; Post, J. Proteomic Identification of Binding Partners for the Brain Metabolite Lanthionine Ketimine (LK) and Documentation of LK Effects on Microglia and Motoneuron Cell Cultures. J. Neuroscience 2010, 30, 2979-2988.
25. Hensley, K. Lanthionine-related compounds for the treatment of inflammatory diseases. U.S. Pat. No. 7,683,055. Issued Mar. 23, 2010.
26. Margiotta, J. F.; Howard, M. J. Eye extract factors promote the expression of acetylcholine sensitivity in chick dorsal root ganglion neurons. Dev. Biol. 1994, 163, 188-201.
27. Hensley, K.; Mhatre, M. C.; Mou, S.; Pye, Q. N.; Stewart, C. A.; West, M. S.; Williamson, K. S. On the relationship of oxidative stress to neuroinflammation: Lessons learned from the G93A-SOD1 mouse model of amyotrophic lateral sclerosis. Antioxid. Redox Signal. 2006, 8, 2075-2087.
28. West, M. S.; Mhatre, M. C.; Ceballos, A.; Floyd, R. A.; Grammas, P.; Gabbita, S. P.; Hamdheydari, L.; Mai, T.; Mou, S.; Pye, Q. N.; Stewart, C. A.; West, S.; Williamson, K. S.; Zemlan, F.; Hensley, K. The arachidonic acid 5-lipoxygenase inhibitor nordihydroguaiaretic acid inhibits TNF□ activation of microglia and extends survival of G93A-SOD1 transgenic mice. J. Neurochem. 2004, 91, 133-143.
29. Charrier, E.; Riebel, S.; Rogemond, V.; Aguer, M.; Thamasset, N.; Honnorat, J. Collapsin response mediator proteins (CRMPs): Involvement in nervous system development and adult neurodegenerative disorders. Mol. Neurobio. 2003, 203, 51-63.
30. Shen, J.; Tareste, D. C.; Paumet, F.; Rothman, J. E.; Melia, T. J. Selective activation of cognate SNAREpins by Sec1/Munc18 proteins. Cell, 2007, 128, 183-195.
31. Chung, C. H.; Kurien, B. T.; Mehta, P.; Mhatre, M.; Mou, S.; Pye, Q. N.; Stewart, C.; West, M.; Williamson, K. S.; Post, J.; Liu, L.; Wang, R.; Henslty, K. Identification of lanthionine synthase C-like protein-1 as a prominent glutathione binding protein expressed in the mammalian central nervous system. Biochemistry 2007, 46, 3262-3269.
32. Zhang, W.; Wang, L.; Liu, Y.; Xu, J.; Zhu, G.; Cang, H.; Li, X.; Bartlam, M.; Hensley, K.; Li, G.; Rao, Z.; Zhang, X. C. Structure of human lanthionine synthetase C-like protein 1 and its interaction with EPS8 and glutathione. Genes Dev. 2009, 23, 1387-1392.
33. Chatterjee, C.; Paul, M.; Xie, L.; van der Donk, W. A. Biosynthesis and mode of action of lantibiotics. Chem. Rev., 2005, 105, 633-683.
34. Willey, J. M.; Van der Donk, W. A. Lanthionines: Peptides of diverse structure and function. Annu. Rev. Microbiol. 2007, 61, 477-501.
35. Lu, P.; Bevan, D. R.; Lewis, S. N.; Bassaganya-Riera, J. Molecular modeling of lanthionine synthetase component C-like protein 2: a potential target for the discovery of novel type 2 diabetes prophylactics and therapeutics. J. Mol. Model. 2010, Epub. Ahead of print, DOI 10.1007/s00894-010-0748-y.
36. Sturla, L.; Fresia, C.; Guida, L.; Bruzzone, S.; Scarfi, S.; Usai, C.; Fruscione, F.; Magnone, M.; Millo, E.; Basile, G.; Grozio, A.; Jacchetti, E.; Allegretti, M.; De Flora, A.; Zocchi, E. LanCL2 is necessary for abscisic acid binding and signaling in human granulocytes and in rat insulinoma cells. J. Biol. Chem. 2009, 284, 28045-28057.
37. Linetsky, M.; Hill, J. M.; LeGrand, R. D.; Hu, F. Dehydroalanine crosslinks in human lens. Exp. Eye Res. 2004, 79, 499-512. Wang, H.; Zhang, J.; Xian, M. Facile formation of dehydroalanine from S-nitrosocysteines. J. Am. Chem. Soc. 2009, 131, 13238-13239.

1. A compound having the structure of Formula I: 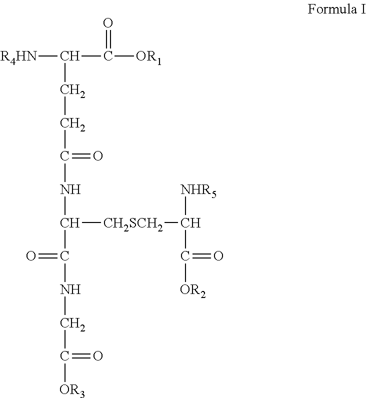
wherein R₁, R₂ and R₃ are each independently selected from the group consisting of: hydrogen, halogen, alkyl, alkenyl, alkynyl, cycloalkyl, cycloalkenyl, substituted alkyl, substituted alkenyl, substituted alkynyl, substituted cycloalkyl, substituted cycloalkenyl, aryl, heteroaryl silyl, heterosilyl, and heterocyclic;
wherein R₄ and R₅ are each independently selected from the group consisting of: —H; —C(═O)—CH₃; —C(═O)—X—NH₂, wherein X is a heteroatom substituted or unsubstituted alkyl or alkynyl, or heteroatom-substituted CN-amido, or
pharmaceutically acceptable salts, hydrates, and optical isomers thereof.

2. The compound of claim 1, wherein R₁, R₂ and R₃ are each —OH.

3. The compound of claim 1, wherein R₄ and R₅ are each NH₂.

4. The compound of claim 1, wherein R₁, R₂ and R₃ are each —OH and wherein R₄ and R₅ are each NH₂.

5. A composition of matter comprising γGlu-Cys(S-Ala)-Gly, and pharmaceutically acceptable salts or hydrates thereof.

6. A method of treating a disease comprising administering to a subject a pharmacologically effective amount of a compound of claim 1.

7. The method of claim 6, wherein the subject is a mammal.

8. The method of claim 6, wherein the subject is a human.

9. The method of claim 6, wherein the method further comprises administering a second anti-inflammatory compound, adjuvant or additional therapeutic to the subject.

10. The method of claim 9, wherein the second anti-inflammatory compound is pyruvate.

11. The method of claim 6, wherein the disease is an inflammatory disease.

12. The method of claim 6, wherein the disease is selected from the group consisting of: sepsis; amyotrophic lateral sclerosis (ALS), a degenerative motor neuron disease, Alzheimer's disease, Parkinson's disease, Huntington's disease, multiple sclerosis, macular degeneration, a cardiovascular disease, atherosclerosis, rheumatoid arthritis; lupus; fibromyalgia; neuropathy; allergy; autoimmune disease; diabetes; ulcerative colitis; inflammatory bowel disease (IBD); hypertension; attention deficit disorder; depression (e.g., major depression); schizophrenia; chronic pain; or generalized anxiety disorder.

13. A method for evaluating the effectiveness of a compound of claim 1 as useful for treatment of an inflammatory disease, the method comprising:
(i) introducing a compound of claim 1 to a macrophage cell, and
(ii) measuring the response of the macrophage cell to an inflammatory stimulus.

14. The method of claim 13, wherein the macrophage cell is a microglial cell.

15. The method of claim 13, wherein the inflammatory stimulus is a pro-inflammatory cytokine.

16. The method of claim 15, wherein the pro-inflammatory cytokine is TNFα or IFNγ.

17. The method of claim 14, wherein nitric oxide production from the macrophage cell is evaluated.

18. The method of claim 17, wherein said evaluation comprises measuring nitrite production from the macrophage cell.

19. A method for evaluating the effectiveness of a compound of claim 1 as useful for treatment of an inflammatory disease, wherein the method comprises:
(i) introducing a compound of claim 1 to a mouse model of inflammation, and
(ii) measuring the response of the mouse model to an inflammatory stimulus.

20. A method of claim 19, wherein the mouse model comprises SOD1-G93A mice.

21. A method of claim 19, wherein the inflammatory stimulus is a pro-inflammatory cytokine.

22. A method of claim 19, wherein the pro-inflammatory cytokine is TNFα or IFNγ.

23. A method of claim 19, wherein onset of paralysis is measured.

24. A method of claim 19, wherein age at death is measured.

25. A method of reducing damage to a cell resulting from oxidative stress and/or excitatory amino acid toxicity, wherein the compound of claim 1 is contacted with the cell, wherein the cell is selected from the group consisting of: a neuron; a macrophage; a glial cell; a motoneuron; an astroglia cell; and a microglial cell; and wherein the glial cell is not a glioma cell.

26. The method of claim 25, wherein the cell is present in a subject.

27. The method of claim 26, wherein the subject is a human patient.

28. A method of treating a subject at risk for having a stroke, comprising administering to the subject a pharmacologically effective amount of claim 1.

29. The method of claim 28, wherein the subject is a human patient.

30. The method of claim 28, wherein the subject has had a stroke.

31. A method of treating a subject with cancer, comprising administering to the subject a pharmacologically effective amount of a compound of claim 1.

32. The method of claim 31, wherein said cancer is brain, lung, liver, spleen, kidney, lymph node, small intestine, pancreas, blood cell, bone, colon, stomach, breast, endometrium, prostate, testicle, ovary, central nervous system, skin, head, and neck, esophagus, or bone marrow cancer.

33. A method of synthesizing a glutathione lanthionine derivative, the method comprising reacting reduced glutathione (GSH) with β-chloroalanine.

34. A pharmaceutical composition comprising a pharmacologically effective amount of a compound of claim 1, and a second anti-inflammatory compound.

35. The pharmaceutical composition of claim 34 wherein the second anti-inflammatory compound comprises pyruvate.

36. The pharmaceutical composition of claim 34, wherein the pharmaceutical composition stimulates endogenous production of lanthionine ketimine (LK).
